(19) United States
(12) Patent Application Publication
(10) Pub. No.: US 2020/0055095 A1
(43) Pub. Date: Feb. 20, 2020

(54) SANITIZING SYSTEM WITH A PLURALITY OF MODULAR TREATMENT STATIONS

(71) Applicant: Michael Ingle, Corinth, TX (US)

(72) Inventor: Michael Ingle, Corinth, TX (US)

(21) Appl. No.: 15/597,440

(22) Filed: Oct. 20, 2017

(51) Int. Cl. B08B 1/02; A61L 2/07

(52) U.S. Cl. CPC B08B 1/02; A61L 2/202; A61L 2/07

(57) ABSTRACT

A sanitizing system has a conveyor system, an elevator system, a control system, a plurality of height sensors, and a plurality of modular treatment stations for sanitizing and cleaning support objects infested with at least one contaminant. The plurality of modular treatment stations include an ultraviolet light treatment station, a steam treatment station, a vacuum treatment station, a heat treatment station, and a pair of ozone generators. The sanitizing system sanitizes and cleans support objects infested with at least one contaminant.

### CROSS REFERENCE TO RELATED APPLICATIONS

The present application is Continuation-In-Part of co-pending U.S. patent application Ser. No. 14/290,113, filed on May 29, 2014, entitled “System and Method for Cleaning and Sanitizing Mattresses.” The present application is also incorporating by reference U.S. patent application Ser. No. 13/507,425, filed on Jun. 27, 2012, entitled “System and Method for Cleaning and Sanitizing Mattresses,” now abandoned. These references are hereby incorporated in their entirety.

### FIELD

The present embodiments generally relates to sanitizing system with a plurality of modular treatment stations for sanitizing and cleaning support objects infested with at least one contaminant.

### BACKGROUND

A need exists for reduce cross contamination from infectious diseases in hospitals.

A further need exists for removing other people's filth from mattresses while staying at a hotel or institution.

A need exists to extend the life of mattresses in hospitals without increasing the need to buy new mattresses to keep health care costs in control.

A need exists to reduce fecal matter and human urine from bed materials.

The present embodiments meet these needs.

### BRIEF DESCRIPTION OF THE DRAWINGS

The detailed description will be better understood in conjunction with the accompanying drawings as follows:

FIG. 1 depicts a block diagram of the sanitizing system according to one or more embodiments.

FIG. 2 is a block diagram of the power distribution panel connected to components of the sanitizing system.

FIG. 3 depicts the sanitizing system mounted to a transport vehicle according to one or more embodiments.

FIG. 4 depicts at transport vehicle mounted ultraviolet light treatment station according to one or more embodiments.

FIG. 5A-5B depicts the heat treatment station according to one or more embodiments.

FIG. 6A-6B depicts a steam treatment station with jet orifices according to one or more embodiments.

FIG. 7 depicts the vacuum treatment station according to one or more embodiments.

The present embodiments are detailed below with reference to the listed Figures.

### DETAILED DESCRIPTION OF THE EMBODIMENTS

Before explaining the present apparatus in detail, it is to be understood that the apparatus is not limited to the particular embodiments and that it can be practiced or carried out in various ways.

The invention relates to a sanitizing system with a plurality of modular treatment stations for sanitizing and cleaning support objects infested with at least one contaminant.

The invention will stop death of humans because the invention irradiates living organisms that cause people to get staph infections and bed stores.

The invention kills the horrible bacteria CDIFF that kills elderly people with weakened immune system.

The invention reduces land fill trash and reduces carbon footprints by killing off bacteria.

The invention allows mattresses to be donated to homeless shelters and the Salvation Army when previously only new mattresses could be donated.

Mattresses can be salvaged from hurricane disasters with this device, which have gotten soaked in a water related incident.

The following terms are used herein:

The term “conveyor system” refers to a generally planar moving system that conveys a support object, such as a mattress in substantially the same plane, or height from ground. The conveyor system is the platform that is raised or lowered by the elevator system.

The term “elevator system” refers to a system which changes the height from ground of a support object, such as a hospital mattress enabling treatment of the support object at different height from ground as the support object moves on the conveyor system. The elevator system is viewed as integral with the conveyor system.

The term “support objects” as used herein refers to object for supporting a portion of a human or animal body. The term includes but is not limited to mattresses and other form fitted fabric cushions, polymer covered and vinyl covered objects used in hospitals and other institutions and vinyl carpet components including carpet tiles. In the example of mattresses, the mattress sizes can include twin, full, queen and king, California king and extra-long mattress and box springs, chairs, mattress pads, gurney of a hospital bed.

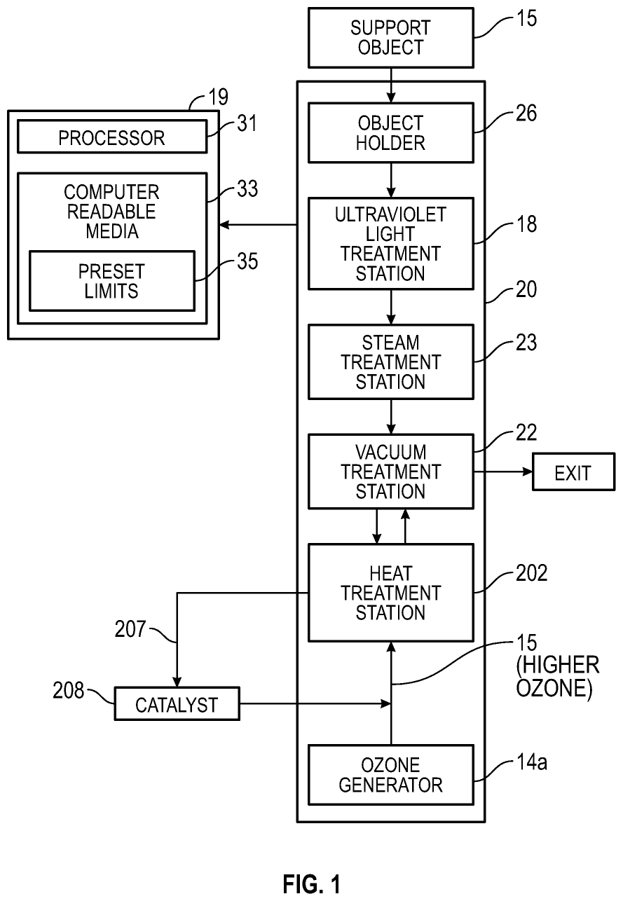

Now turning to the Figures, FIG. 1 depicts a block diagram of the sanitizing system 10 with a plurality of modular treatment stations for sanitizing and cleaning support objects infested with at least one contaminant.

The sanitizing system has conveyor system 8. The conveyer system has an object holder 26 for holding a support object 15, mounted to a conveyor track as the support object 15 moves along the conveyor system.

The sanitizing system 10 includes a control system 19 mounted to the frame and connected to the power distribution panel. The control system includes a processor 31 and a computer readable media 33 with preset limits 35 to actuate movement by the conveyor system and elevator system when a support object is detected.

The sanitizing system 10 receives a support object 15 and transfers the support object 15 through a plurality of modular treatment stations 20.

The plurality of modular treatment stations 20 are mounted in the frame of the sanitizing system and include an ultraviolet light treatment station 18, a steam treatment station 23, a vacuum treatment station 22, a heat treatment station 202, and a pair of ozone generators 14a.

In embodiments, the sanitizing system 10 has an ozone recycle conduit 207 connected to the heat treatment station 202 to suction air with the ozone from the heat treatment station and flow air with the ozone across a catalyst 208 forming air with reduced ozone concentration that is recombined with ozone from the ozone generator.

In embodiments, the support object is sanitized in 1 hour or less from introduction to the frame with a 99.9% kill rate for contaminates on the surface of the support object.

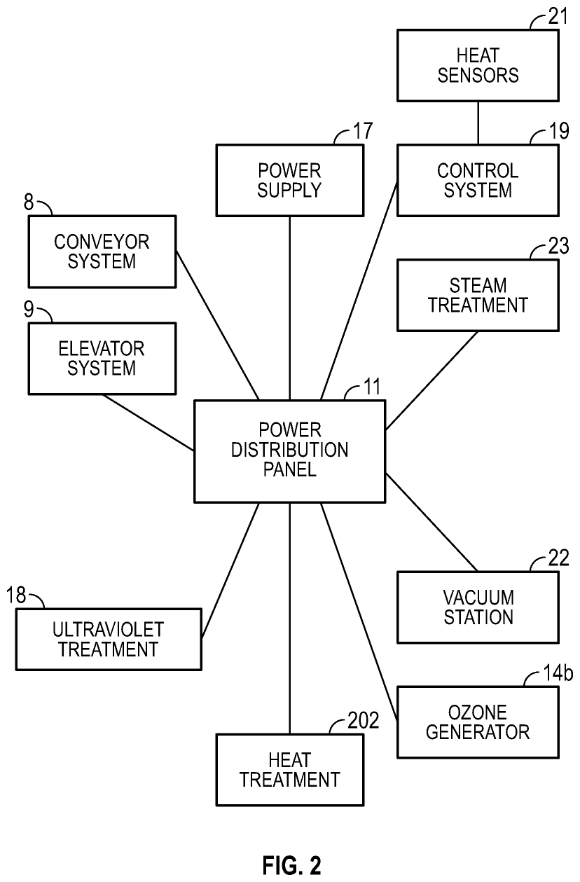

FIG. 2 is a block diagram of the power distribution panel 11, connected to components of the sanitizing system.

The power distribution panel 11 mounted in the frame for receiving electrical power from a power supply 17.

In embodiments, the power distribution panel is connected to the conveyor system 8, the elevator system 9, the ultraviolet light treatment station 18, the heat treatment station 202, the at least one ozone generator 14b, the vacuum treatment station 22, the steam treatment station 23, the control system 19, and a plurality of height sensors 21.

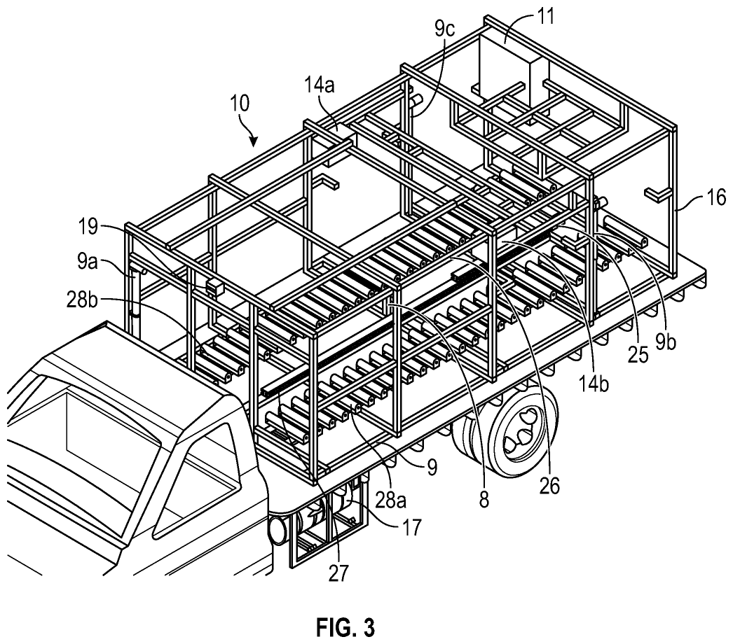

FIG. 3 depicts an overview of the sanitizing system 10 according to one or more embodiments.

The sanitizing system 10 has a frame 16 with a power distribution panel 11 mounted in the frame 16 for receiving electrical power. The power distribution panel is connected to a power supply 17.

In embodiments, the frame 16 can be 14 feet long×8 feet wide by 6 feet tall. The frame 16 may be designed to fit in a shipping container.

A conveyor system 8 is mounted in the frame 16 for receiving a support object and transferring the support object sequentially through a plurality of modular treatment stations. Also, the conveyor system 8 is connected to the power distribution panel 11.

The conveyor system includes a conveyor track 27, an object holder 26 mounted to the conveyor track 27 for containing the support object as the support object moves along the conveyor system 8, and a screw drive 25 connected to the power distribution panel and the object holder for moving the support object from one end of the conveyor track 27 to an opposite end of the conveyor track 27.

A control system 19 is mounted to the frame and connected to the power distribution panel 11 for actuating movement by the conveyor system and elevator system when a support object is detected.

In embodiments, a plurality of height sensors 21 electrically and electronically connected to the control system 19 and mounted to the frame to detect the presence of a support object.

In embodiments, two parallel rows of rollers 28a and 28b mounted on either side of the conveyor track 27.

The elevator system 9a-9c is mounted in the frame 16 and connected to the power distribution panel 11, supporting the conveyor system 8. The elevator system 9 is configured to raise and lower the support object as the support object travels sequentially through a plurality of modular treatment stations 20.

Two ozone generator 14a and 14b are shown mounted to the frame 16 and connected to the power distribution panel 11 and fluidly connected to the heat treatment station.

The ozone generator 14a and 14b induces ozone into the support object in the heat chamber, using a concentration of 10 ppm to 30 ppm ozone based on a total ozone concentration in the heat chamber.

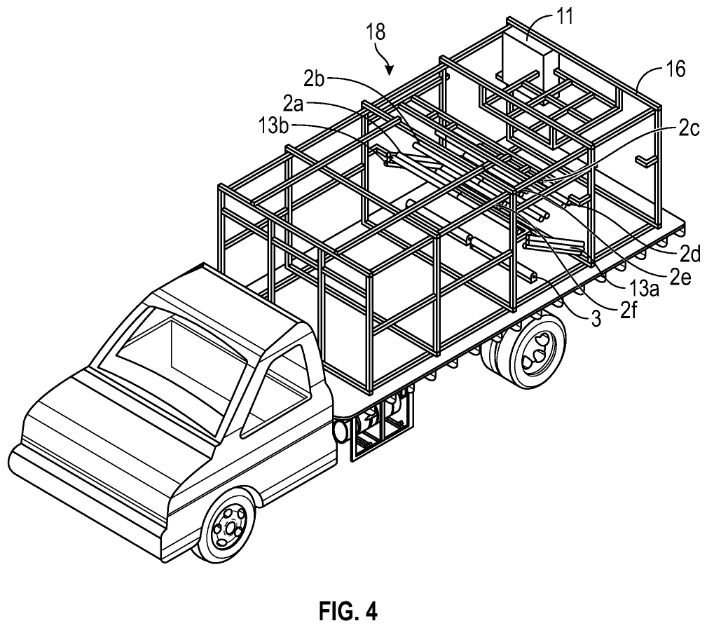

FIG. 4 depicts the ultraviolet light treatment station 18 according to one or more embodiments.

In embodiments, an ultraviolet light treatment station 18 is connected to the power distribution panel 11.

The ultraviolet light treatment station 18 includes an upper ultraviolet light assembly 2a-2f, a lower ultraviolet light assembly 3, a first side ultraviolet light assembly 13a, and a second side ultraviolet light assembly 13b.

The ultraviolet light treatment station surrounds the support object with ultraviolet light in wavelengths from 200 to 290, such as 254 nanometers.

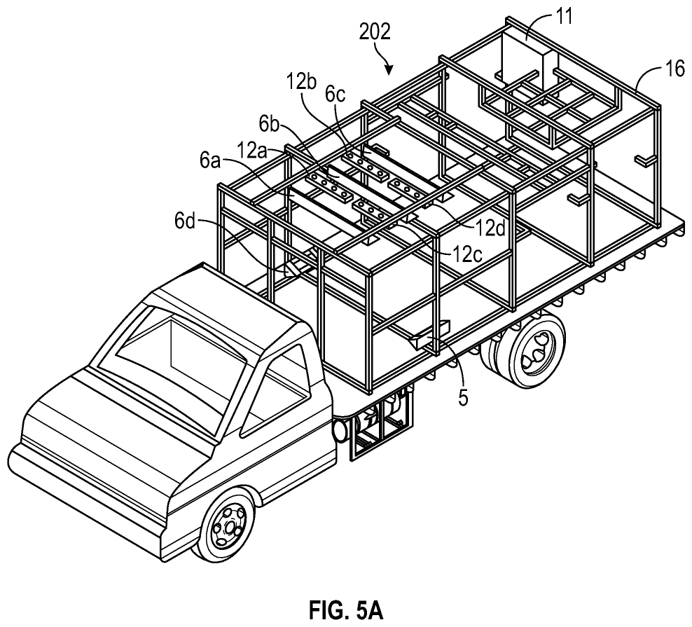

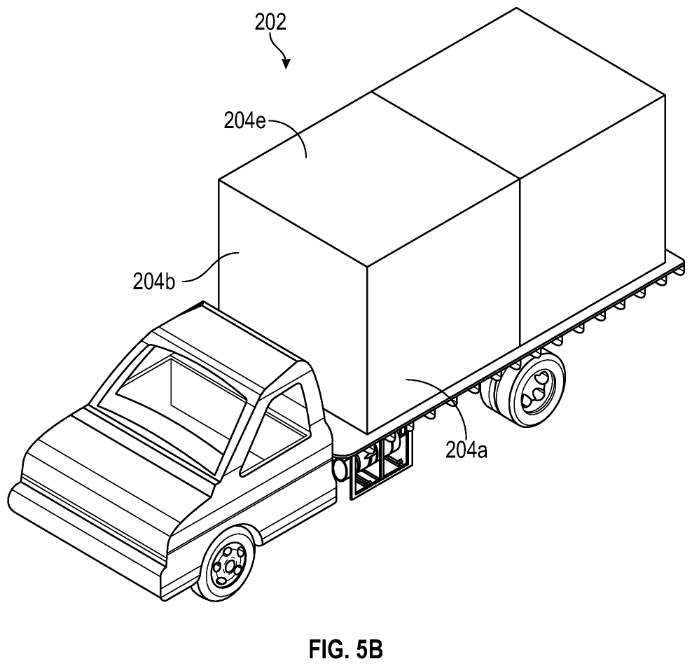

FIG. 5A-5B depicts the heat treatment station according to one or more embodiments.

The heat treatment station 202 is mounted to the frame 16 in series with the ultraviolet light treatment station and connected to the power distribution panel 11.

In embodiments, the heat treatment station 202 generates heat from 90 degrees to 225 degrees Fahrenheit to raise a surface temperature of the support object.

The heat treatment station 202 contains a plurality of upper infrared heaters 6a-6c and a plurality of lower infrared heater 5a-5b mounted to the frame.

In embodiments, the upper infrared heaters 6a-6c can contain three to four infrared heaters.

In embodiments, the lower infrared heater 5a-5b can contain at least two infrared heaters.

A plurality of convection fans 12a-12d are mounted to the frame adjacent the upper and lower infrared heaters.

In embodiments, the plurality of convection fans 12a-12d can include from two to eight convection fans.

A plurality of insulated panels 204a-204e are installed around the heaters and fans forming the heat chamber, wherein the plurality of heaters increase air temperature in the heat chamber and the plurality of convection fans evenly distribute heated air in the heat chamber.

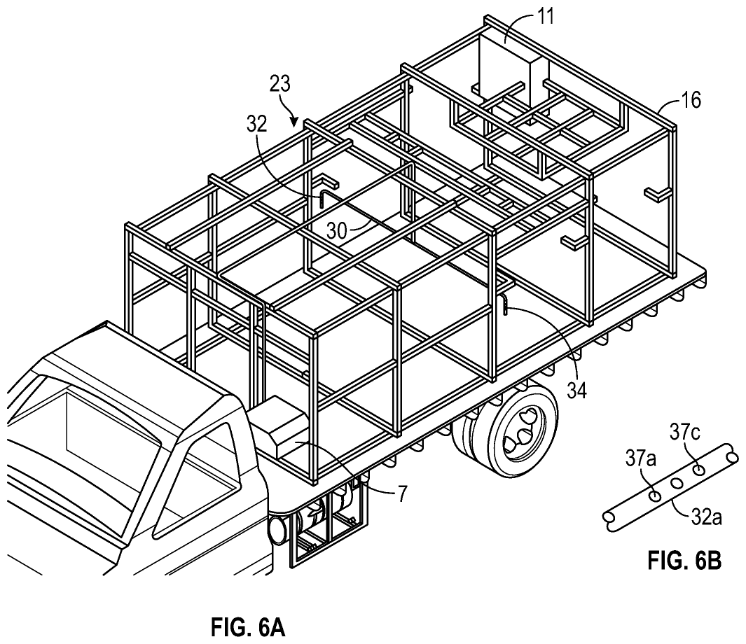

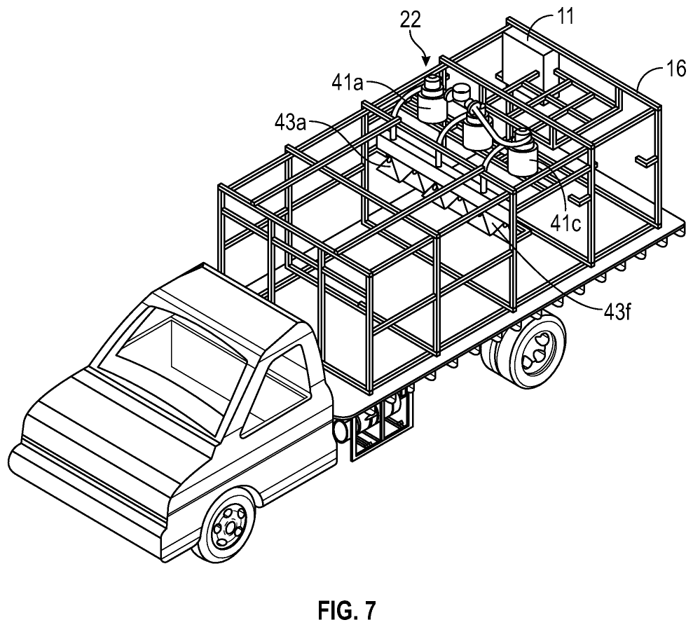

FIG. 6A-6B depicts a steam treatment station 23 connected to a steam generator 7 and connected to the power distribution panel 11 mounted to the frame 16 in series with the ultraviolet light treatment station. The steam treatment station 23 is configured to direct steam at the support object through a plurality of jets.

The steam treatment station contains a top steam jet assembly 30, a first side steam jet assembly 32, and a second side steam jet assembly 34.

In embodiments, the plurality of jets range from 6 to 40 with each jet having a diameter jet orifice 37a-37c from 1/32 inch to one-fourth inch.

FIG. 7 depicts a vacuum treatment station 22 according to one or more embodiments.

The vacuum treatment station 22 is connected to the power distribution panel 11 and mounted to the frame 16 in series with the steam treatment station.

The vacuum treatment station contains a plurality of vacuums 41a-41c connected to a plurality of moveable orifices 43a-43f configured to suction debris from the support object and air surrounding the support object.

### EXAMPLES

### Example 1

A customer, ABC Furniture, sells gently used mattress without a waterproof covering. ABC Furniture has a shipment of mattresses and pillows, which may be contaminated with at least one of viruses, fungi, mold, dust mites, bed bugs, lice and bacteria.

Sleepytime, owns a sanitizing system with a plurality of modular treatment stations for sanitizing and cleaning support objects infested with at least one contaminant. The sanitizing system has a frame with a power distribution panel mounted in the frame for receiving electrical power.

The power distribution panel is turned on to receive the mattresses and pillows on a conveyor system.

The conveyor system, mounted in the frame and connected to the power distribution panel, receives each mattress and pillow individually and transfers the mattresses and pillows through a plurality of modular treatment stations.

An elevator system, mounted in the frame and connected to the power distribution panel, supports the conveyor system. The elevator system raises and lowers the mattresses and pillows as the mattresses and pillows travels sequentially through the plurality of modular treatment stations.

The conveyor moves each mattress and pillow through an ultraviolet light treatment station connected to the power distribution panel. The UV light treatment station surrounds each mattress and pillow with UV light in wavelengths of 254 nanometers.

Next, each mattress and pillow is moved through a heat treatment station, mounted to the frame in series with the ultraviolet light treatment station and connected to the power distribution panel. The heat treatment station generates heat at 165 degrees Fahrenheit to raise a surface temperature of each mattress and pillow.

Each mattress and pillow is then moved to an ozone generator, mounted to the frame, fluidly connected to a heat treatment station, and connected to the power distribution panel. The ozone generator inducing ozone into each mattress and pillow in the heat station, using a concentration of 25 ppm, ozone based on a total ozone concentration in the heat chamber.

Each mattress and pillow is sanitized in 1 hour or less from introduction to the frame with a 99.9% kill rate for contaminates on the surface of the support object.

### Example 2

A homeowner has a mattress with a waterproof covering that has been contaminated with bed bugs.

Sleepytime, owns a sanitizing system with a plurality of modular treatment stations for sanitizing and cleaning support objects infested with at least one contaminant. The sanitizing system has a frame with a power distribution panel mounted in the frame for receiving electrical power.

The power distribution panel is turned on to receive the mattress on a conveyor system.

The conveyor system, mounted in the frame and connected to the power distribution panel, receives the mattress individually and transfers the mattress through a plurality of modular treatment stations.

An elevator system, mounted in the frame and connected to the power distribution panel, supports the conveyor system. The elevator system raises and lowers the mattress as the mattress travels sequentially through the plurality of modular treatment stations.

The conveyor moves the mattress through an ultraviolet light treatment station connected to the power distribution panel. The UV light treatment station surrounds the mattress with UV light in wavelengths of 200 nanometers.

Next, the mattress is moved through a heat treatment station, mounted to the frame in series with the ultraviolet light treatment station and connected to the power distribution panel. The heat treatment station generates heat at 97 degrees Fahrenheit to raise a surface temperature of the mattress.

The conveyer then moves the mattress through the steam treatment station, mounted to the frame in series with the ultraviolet light treatment station and connected to a steam generator and power distribution panel. The steam treatment station directs steam at the mattress through a plurality of jets. The steam can have a moisture concentration from three to five percent.

Afterwards, the mattress is moved to a vacuum treatment station, connected to the power distribution panel and mounted to the frame in series with the steam treatment station. The vacuum treatment station has a plurality of vacuums each having a plurality of moveable orifices, which suction debris from the mattress and the air surrounding each mattress and pillow. The combination of vacuums in the vacuum treatment station can operate at 2150 air watts.

Each mattress and pillow is then moved to an ozone generator, mounted to the frame, fluidly connected to a heat treatment station, and connected to the power distribution panel. The ozone generator inducing ozone into each mattress and pillow in the heat station, using a concentration of 10 ppm ozone based on a total ozone concentration in the heat chamber.

Each mattress and pillow is sanitized in 1 hour or less from introduction to the frame with a 99.9% kill rate for contaminates on the surface of the support object.

### Example 3

A homeowner has a mattress that has been contaminated with bed bugs.

XYZ Company owns a sanitizing system with a plurality of modular treatment stations for sanitizing and cleaning support objects infested with at least one contaminant. The sanitizing system has a frame with a power distribution panel mounted in the frame for receiving electrical power.

The power distribution panel is turned on to receive the mattress on a conveyor system.

The conveyor system, mounted in the frame and connected to the power distribution panel, receives the mattress individually and transfers the mattress through a plurality of modular treatment stations.

An elevator system, mounted in the frame and connected to the power distribution panel, supports the conveyor system. The elevator system raises and lowers the mattress as the mattress travels sequentially through the plurality of modular treatment stations.

The conveyor moves the mattress through an ultraviolet light treatment station connected to the power distribution panel. The UV light treatment station surrounds the mattress with UV light in wavelengths of 290 nanometers.

Next, the mattress is moved through a heat treatment station, mounted to the frame in series with the ultraviolet light treatment station and connected to the power distribution panel. The heat treatment station generates heat at 195 degrees Fahrenheit to raise a surface temperature of the mattress.

Afterwards, the mattress is moved to a vacuum treatment station, connected to the power distribution panel and mounted to the frame in series with the steam treatment station. The vacuum treatment station has a plurality of moveable orifices, which suction debris from the mattress and the air surrounding each mattress and pillow.

Each mattress and pillow is then moved to an ozone generator, mounted to the frame, fluidly connected to a heat treatment station, and connected to the power distribution panel. The ozone generator inducing ozone into each mattress and pillow in the heat station, using a concentration of 30 ppm ozone based on a total ozone concentration in the heat chamber.

An ozone recycle conduit is connected to the heat treatment station to suction air with the ozone from the heat treatment station and flow air with the ozone across a catalyst forming air with reduced ozone concentration that is recombined with ozone from the ozone generator.

Each mattress and pillow is sanitized in 1 hour or less from introduction to the frame with a 99.9% kill rate for contaminates on the surface of the support object.

In embodiments, it is contemplated to screw jet nozzles into orifices to increase treatment effectiveness while reducing treatment time.

While these embodiments have been described with emphasis on the embodiments, it should be understood that within the scope of the appended claims, the embodiments might be practiced other than as specifically described herein.

What is claimed is:

1. A sanitizing system with a plurality of modular treatment stations for sanitizing and cleaning support objects infested with at least one contaminant, the sanitizing system comprising:
a. a frame;
b. a power distribution panel mounted in the frame for receiving electrical power from a power supply;
c. a conveyor system mounted in the frame for receiving a support object and transferring sequentially the support object through a plurality of modular treatment stations, the conveyor system connected to the power distribution panel;
d. an elevator system mounted in the frame and connected to the power distribution panel supporting the conveyor system, the elevator system configured to raise and lower the conveyor system;
e. a control system mounted to the frame and connected to the power distribution panel comprising a processor and a computer readable media with preset limits to actuate movement by the conveyor system and elevator system when a support object is detected;
f. a plurality of height sensors electrically and electronically connected to the control system and mounted to the frame to detect the presence of the support object;
g. a plurality of modular treatment stations mounted in the frame connected to the control system and the power distribution panel, the modular treatment stations sequentially actuated by the control system when one of the height sensors of the plurality of height sensors mounted to the frame signals the presence of the support object in a treatment module of the plurality of treatment modules, the plurality of modular treatment stations comprising:
(i) an ultraviolet light treatment station mounted to the frame, the ultraviolet light treatment station surrounding the support object with ultraviolet light in wavelengths from 200 to 290 nanometers;
(ii) a heat treatment station mounted to the frame in series with the ultraviolet light treatment station generating heat from 90 degrees to 195 degrees Fahrenheit to raise a surface temperature of the support object from ambient temperature to 165 degrees Fahrenheit at a rate between 10 to 20 degrees per minute; and
(iii) at least one ozone generator mounted to the frame for inducing ozone onto and optionally into the support object in the heat treatment station, using a concentration of from 10 ppm to 25 ppm ozone based on a total ozone concentration in the heat treatment station, wherein the support object is sanitized in 1 hour or less from placement on the frame with a 99.9% kill rate for contaminates on and optionally into the support object, wherein the sanitizing system sanitizes and cleans support objects infested with at least one contaminant.

2. The sanitizing system of claim 1, wherein the ultraviolet light treatment station comprises:
a. an upper ultraviolet light assembly;
b. a lower ultraviolet light assembly;
c. a first side ultraviolet light assembly; and
d. a second side ultraviolet light assembly.

3. The sanitizing system of claim 1, wherein the heat treatment station comprises:
a. a plurality of upper infrared heaters and a plurality of lower infrared heaters mounted to the frame;
b. a plurality of convection fans mounted to the frame adjacent the plurality of upper and the plurality of lower infrared heaters; and
c. a plurality of insulated panels installed around the plurality of upper and the plurality of lower infrared heaters and the plurality of convection fans; wherein the plurality of upper and the plurality of lower infrared heaters increase air temperature around the support object and the plurality of convection fans evenly distribute heated air around the support object.

4. The sanitizing system of claim 1, comprising a steam treatment station as one of the modular treatment stations, the steam treatment station connected to a steam generator and connected to the power distribution panel mounted to the frame in series with the ultraviolet light treatment station, the steam treatment station configured to direct steam at the support object.

5. The sanitizing system of claim 4, wherein the steam treatment station comprises:
a. a top steam jet assembly;
b. a first side steam jet assembly; and
c. a second side steam jet assembly, each steam jet assembly for directing steam at the support object.

6. The sanitizing system of claim 1, comprising a vacuum treatment station connected to the power distribution panel and mounted to the frame in series with the steam treatment station, the vacuum treatment station comprising: a plurality of vacuums connected to a plurality of moveable orifices configured to suction debris from the support object and air surrounding the support object.

7. The sanitizing system of claim 1, comprising an ozone recycle conduit connected to the heat treatment station to suction air with the ozone from the heat treatment station and flow air with the ozone across a catalyst forming air with reduced ozone concentration that is recombined with ozone from the ozone generator.

8. The sanitizing system of claim 1, the conveyor system comprising:
a. a conveyor track supported by the elevator system;
b. two parallel rows of rollers mounted on either side of the conveyor track;
c. an object holder for holding a support object, mounted to the conveyor track as the support object moves along the conveyor system; and
d. a screw drive connected to the power distribution panel and the object holder for moving the support object from one end of the conveyor track to an opposite end of the conveyor track.

9. The sanitizing system of claim 5, wherein each jet assembly has from 6 to 40 jet orifices, with each jet orifice having a diameter from 1/32 inch to one-fourth inch.

10. The sanitizing system of claim 1, wherein the contaminates are at least one of: a virus, a fungus, a mold, dust mites, bed bugs, lice, urine, sweat, dirt, odor, smoke, fire, water, and bacteria.
